(19) United States
(12) Patent Application Publication
(10) Pub. No.: US 2005/0171360 A1
(43) Pub. Date: Aug. 4, 2005

(54) Preparation of n-methyl-3-hydroxy- 3-(2-thienyl)propylamine via novel thiophene derivatives containing carbamate groups as intermediates

(76) Inventors: Dietmar Reichert, Eschau (DE); Juan Jose Almena Perea, Hanau (DE); Michael Schwarm, Alzenau (DE); Karlheinz Drauz, Freigericht (DE); Hans-Peter Krimmer, Pittenhart (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/503,600

(22) PCT Filed: Jan. 30, 2003
(86) PCT No.: PCT/EP03/00910

(30) Foreign Application Priority Data
Feb. 22, 2002 (DE) 102-07-586.7

(51) Int. Cl. C07D 333/22

(52) U.S. Cl. 549/70

(57) ABSTRACT

A novel route is described for the synthesis of N-methyl-3-hydroxy-3-(2-thienyl)propylamine IV, which can be used as a starting compound for the preparation of duloxetine. N-methyl-3-hydroxy-3-(2-thienyl)propylamine is synthesized via novel thiophene derivatives containing carbamate groups, I and IIa, as intermediates.

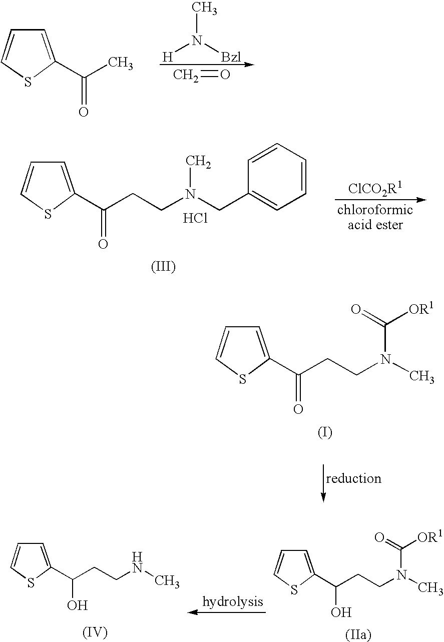

The present invention describes a novel route for the synthesis of N-methyl-3-hydroxy-3-(2-thienyl)propylamine, which can be used as a starting compound for the preparation of duloxetine. N-methyl-3-hydroxy-3-(2-thienyl)propylamine is synthesized via novel thiophene derivatives containing carbamate groups as intermediates.

Duloxetine, or (S)-(+)-N-methyl-3-(1-naphthyloxy)-3-(2-thienyl)propylamine hydrochloride, is a pharmaceutical used as an antidepressant and for the treatment of urinary incontinence. It inhibits the uptake of both norepinephrine and serotonin. The synthesis of duloxetine is described in detail in EP-A-273 658, EP-A-457 559 and EP-A-650 965.

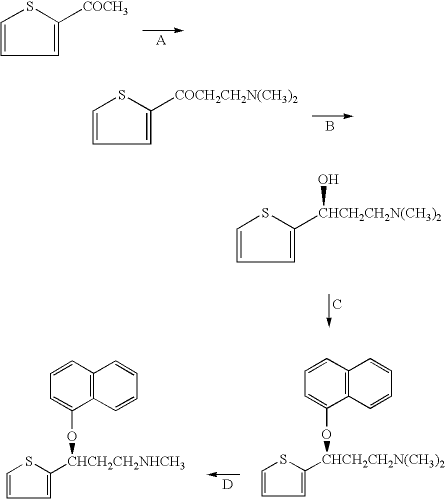

Starting from 2-acetylthiophene, an aminomethylation with dimethylamine and formaldehyde (Mannich reaction) is carried out in step A. The 3-dimethylamino-1-(2-thienyl)-1-propanone formed is reduced to the corresponding alcohol, 1-hydroxy-1-(2-thienyl)-3-dimethylaminopropane, by means of complex hydrides in step B. The alcohol is then converted in step C with an alkali metal hydride and 1-fluoro-naphthalene, optionally in the presence of a potassium compound (cf. EP-A-650 965), to the naphthyl derivative, N,N-dimethyl-3-(1-naphthyloxy)-3-(2-thienyl)propylamine. In the final step, D, the amino group is then demethylated by reaction with a chloroformic acid ester, preferably phenyl chloroformate or trichloroethyl chloroformate, optionally in the presence of a mixture of zinc and formic acid (EP-A-457 559), followed by alkaline hydrolysis of the carbamate to give N-methyl-3-(1-naphthyloxy)-3-(2-thienyl)propylamine. The desired duloxetine is the (S)-(+) enantiomer of the product in the hydrochloride form.

As the above synthesis of N-methyl-3-(1-naphthyloxy)-3-(2-thienyl)propylamine usually produces a racemate, special measures are necessary to prepare the (S)-(+) enantiomer specifically. Thus EP-A-457 559 discloses an asymmetric reduction in step B by means of a complex of lithium aluminium hydride and a chiral ligand.

One particular disadvantage of the synthetic route described above is the demethylation of step D. Here, in the last stage of the synthesis of a drug, chloroformic acid esters with a strong caustic action are used, optionally in combination with toxic zinc, and carcinogenic methyl chloride is released. Expensive separation and purification steps are then consequently essential. It would therefore be desirable to convert the dimethylamino group to the desired monomethylamino group in an earlier step of the synthesis. An alternative synthetic route for duloxetine would proceed via the conversion of (S)—N-methyl-3-hydroxy-3-(2-thienyl)propylamine to (S)-(+)-N-methyl-3-(1-naphthyloxy)-3-(2-thienyl)propylamine in the last step.

Example 2 of EP-A-457 559 describes the enantioselective reduction of 3-N-benzylmethyl-1-(2-thienyl)-1-propanone to N-methyl-N-benzyl-3-(β-hydroxy)-3-(2-thienyl)propylamine. However, there is no indication as to how N-methyl-N-benzyl-3-(β-hydroxy)-3-(2-thienyl)propylamine can be debenzylated. Studies carried out by the inventors of the present patent application have shown that the reaction of N-methyl-N-benzyl-3-hydroxy-3-(2-thienyl)propylamine with hydrogen in the presence of conventional palladium catalysts, in solvents such as alcohols and acetic acid, does not yield the desired debenzylated monomethylamine, N-methyl-3-hydroxy-3-(2-thienyl)propylamine.

The object of the present invention is therefore to provide a simple route for the synthesis of N-methyl-3-hydroxy-3-(2-thienyl)propylamine via isolatable intermediates which also allows the preparation of optically active N-methyl-3-hydroxy-3-(2-thienyl)propylamine.

The present invention provides on the one hand the thiophene derivatives containing carbamate groups of formulae I and II: 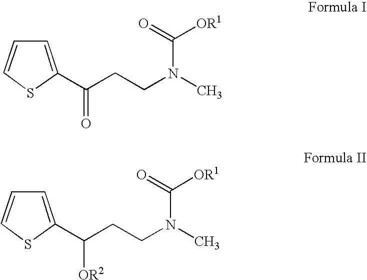
as intermediates, in which R¹ is selected from hydrogen; aliphatic, cycloaliphatic and aromatic hydrocarbon groups which can be substituted by one or more substituents containing heteroatoms; mixed aliphatic-cycloaliphatic hydrocarbon groups which can be substituted by one or more substituents containing heteroatoms; mixed aliphatic-aromatic hydrocarbon groups which can be substituted by one or more substituents containing heteroatoms; and mixed cycloaliphatic-aromatic hydrocarbon groups which can be substituted by one or more substituents containing heteroatoms; and R² in formula II is selected from hydrogen; branched or unbranched, saturated or unsaturated acyl groups, preferably having 1-10 C atoms, which can be substituted by one or more substituents containing heteroatoms, preferably halogen or alkoxy radicals preferably having 1-5 C atoms; aromatic acyl groups, preferably having 6-12 C atoms, which can be substituted by one or more substituents containing heteroatoms, preferably halogen or alkoxy radicals preferably having 1-5 C atoms; sulfonylalkyl groups preferably having 1-5 C atoms; sulfonylalkenyl groups preferably having 2-5 C atoms; and sulfonylaryl groups preferably having 6-14 C atoms.

In preferred embodiments R¹ is selected from branched or unbranched alkyl groups preferably having 1-10 C atoms; branched or unbranched alkenyl groups preferably having 2-10 C atoms; branched or unbranched alkynyl groups preferably having 2-10 C atoms; halogen-, nitro- or alkoxy-substituted alkyl groups, preferably halogen-substituted or C₁- to C₅-alkoxy-substituted alkyl groups; halogen-, nitro- or alkoxy-substituted alkenyl groups, preferably halogen-substituted or C₁- to C₅-alkoxy-substituted alkenyl groups; halogen-, nitro- or alkoxy-substituted alkynyl groups, preferably halogen-substituted or C₁- to C₅-alkoxy-substituted alkynyl groups; a benzyl group; a halogen-, nitro- or alkoxy-substituted benzyl group, preferably a halogen-substituted or C₁- to C₅-alkoxy-substituted benzyl group; a phenyl group; a halogen-, nitro- or alkoxy-substituted phenyl group, preferably a halogen-substituted or C₁- to C₅-alkoxy-substituted phenyl group; a fluorenylalkyl group; a menthyl, fenchyl or cholesteryl group; and a hydroxybornyl group.

Particularly preferably, R¹ is selected from the methyl, ethyl, propyl, benzyl, phenyl, isobutyl and menthyl radicals.

In one preferred embodiment of the present invention, R² in the thiophene derivative of formula II is hydrogen and the compound is thus represented by formula IIa below: 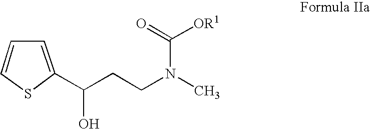
in which R¹ is as defined above.

Examples of thiophene derivatives of formula I are:
3-N-methoxycarbonyl-N-methylamino-1-(2-thienyl)-1-propanone,
3-N-ethoxycarbonyl-N-methylamino-1-(2-thienyl)-1-propanone,
3-N-benzyloxycarbonyl-N-methylamino-1-(2-thienyl)-1-propanone,
3-N-isobutoxycarbonyl-N-methylamino-1-(2-thienyl)-1-propanone,
3-N-allyloxycarbonyl-N-methylamino-1-(2-thienyl)-1-propanone,
3-N-(2-benzyloxyethyl)oxycarbonyl-N-methylamino-1-(2-thienyl)-1-propanone,
3-N-(2-bromoethyl)oxycarbonyl-N-methylamino-1-(2-thienyl)-1-propanone,
3-N-(4-bromophenyl)oxycarbonyl-N-methylamino-1-(2-thienyl)-1-propanone,
3-N-(3-butenyl)oxycarbonyl-N-methylamino-1-(2-thienyl)-1-propanone,
3-N-(2-butynyl)oxycarbonyl-N-methylamino-1-(2-thienyl)-1-propanone,
3-N-butoxycarbonyl-N-methylamino-1-(2-thienyl)-1-propanone,
3-N-(4-chlorobutyl)oxycarbonyl-N-methylamino-1-(2-thienyl)-1-propanone,
3-N-(1-chloroethyl)oxycarbonyl-N-methylamino-1-(2-thienyl)-1-propanone,
3-N-(2-chloroethyl)oxycarbonyl-N-methylamino-1-(2-thienyl)-1-propanone,
3-N-(1-chloromethylpropyl)oxycarbonyl-N-methylamino-1-(2-thienyl)-1-propanone,
3-N-(2-chlorophenyl)oxycarbonyl-N-methylamino-1-(2-thienyl)-1-propanone,
3-N-(4-chlorophenyl)oxycarbonyl-N-methylamino-1-(2-thienyl)-1-propanone,
3-N-(3-chlorophenyl)oxycarbonyl-N-methylamino-1-(2-thienyl)-1-propanone,
3-N-neopentoxycarbonyl-N-methylamino-1-(2-thienyl)-1-propanone,
3-N-dodecyloxycarbonyl-N-methylamino-1-(2-thienyl)-1-propanone,
3-N-(2-ethylhexyl)oxycarbonyl-N-methylamino-1-(2-thienyl)-1-propanone,
3-N-(9-fluorenyl)methoxycarbonyl-N-methylamino-1-(2-thienyl)-1-propanone,
3-N-(9-fluorenyl)ethoxycarbonyl-N-methylamino-1-(2-thienyl)-1-propanone,
3-N-hexyloxycarbonyl-N-methylamino-1-(2-thienyl)-1-propanone,
3-N-isoprenyloxycarbonyl-N-methylamino-1-(2-thienyl)-1-propanone,
3-N-isopropoxy-N-methylamino-1-(2-thienyl)-1-propanone,
3-N-menthyloxycarbonyl-N-methylamino-1-(2-thienyl)-1-propanone,
3-N-(4-methoxyphenyl)oxycarbonyl-N-methylamino-1-(2-thienyl)-1-propanone,
3-N-(4-nitrophenyl)oxycarbonyl-N-methylamino-1-(2-thienyl)-1-propanone,
3-N-octyloxycarbonyl-N-methylamino-1-(2-thienyl)-1-propanone,
3-N-(2-propynyl)oxycarbonyl-N-methylamino-1-(2-thienyl)-1-propanone,
3-N-propoxycarbonyl-N-methylamino-1-(2-thienyl)-1-propanone,
3-N-(2,2,2-trichloro-tert-butyl)oxycarbonyl-N-methylamino-1-(2-thienyl)-1-propanone,
3-N-(2,2,2-trichloroethyl)oxycarbonyl-N-methylamino-1-(2-thienyl)-1-propanone and
3-N-vinyloxycarbonyl-N-methylamino-1-(2-thienyl)-1-propanone.

Examples of thiophene derivatives of formula IIa are the alcohols corresponding to the above compounds.

The present invention further relates to a process for the preparation of the thiophene derivative of formula I, comprising (i) the aminomethylation of 2-acetylthiophene with N-methylbenzylamine and formaldehyde in the presence of an acid to form 3-N-benzylmethylamino-1-(2-thienyl)-1-propanone of formula III: 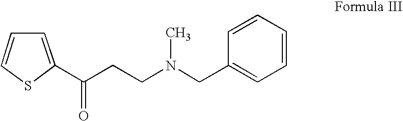
and then (ii) the reaction of 3-N-benzylmethylamino-1-(2-thienyl)-1-propanone with a chloroformic acid ester ClCO₂R¹, in which R¹ is as defined above, in the presence of a base, and to a process for the preparation of the thiophene derivative of formula IIa by reduction of the thiophene derivative of formula I.

The preparation of N-methyl-3-hydroxy-3-(2-thienyl)-propylamine of formula IV: 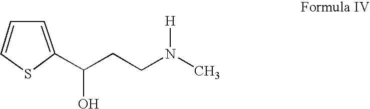
by hydrolysis of the thiophene derivative of formula IIa, and the use of the thiophene derivatives of formulae I and IIa in the synthesis of N-methyl-3-(1-naphthyloxy)-3-(2-thienyl)propylamine, are also claimed.

Overall, the synthesis of N-methyl-3-hydroxy-3-(2-thienyl)-propylamine via the thiophene derivatives I and IIa can be illustrated by Scheme 1 below:

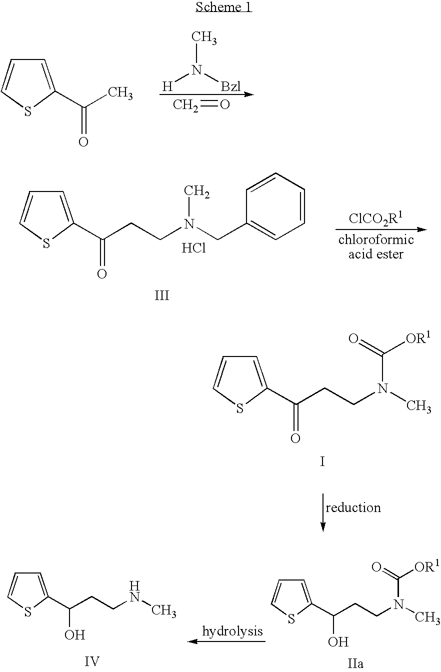

In the first step, 3-N-benzylmethylamino-1-(2-thienyl)-1-propanone of formula III can be prepared from 2-acetylthiophene, in a manner known to those skilled in the art, by aminomethylation with N-methylbenzylamine and formaldehyde (Mannich reaction) in the presence of an acid, preferably hydrochloric acid, sulfuric acid, methanesulfonic acid or an acidic ion exchanger, in conventional solvents, preferably water, methanol, ethanol, n-propyl alcohol, isopropyl alcohol, butanol or mixtures thereof. It is advantageous to use a 10 to 50% excess of both N-methylbenzylamine and formaldehyde. It is particularly preferred to use an approximately 20% excess of both N-methylbenzylamine and formaldehyde. The reaction temperature is preferably 50 to 100° C. and the reaction time is conventionally 3 to 30 h. Mannich reactions of 2-acetylthiophene with dimethylamine or N-methylbenzylamine are described e.g. in Examples 1 and 2 of EP-A-457 559.

The reaction of 3-N-benzylmethylamino-1-(2-thienyl)-1-propanone of formula III with a chloroformic acid ester, preferably methyl, ethyl, propyl, benzyl, phenyl, isobutyl or menthyl chloroformate, in a solvent such as toluene, xylene, benzene, dichlorobenzene, 1,2-dichloroethane, chloroform, methylene chloride, THF, dioxane, diglyme, ethyl acetate, butyl acetate, a formic acid ester or mixtures thereof, in the presence of suitable bases, e.g. triethylamine, diisopropylethylamine (Hünig base), 1,4-diazabicyclo[2.2.2]octane (DABCO®), 1,5-diazabicyclo-[4.3.0]non-5-ene (DBU), pyridine, sodium carbonate, potassium carbonate, sodium hydrogencarbonate, potassium hydrogencarbonate or mixtures thereof, gives the corresponding thiophene derivative of general formula I in which the amino group is protected by a carbamate group, with the elimination of benzyl chloride. The molar ratio of 3-N-benzylmethylamino-1-(2-thienyl)-1-propanone to chloroformic acid ester is preferably 1:0.8 to 1:5 and particularly preferably 1:1.5 to 1:3. The base is advantageously used in an equimolar amount relative to the 3-N-benzylmethylamino-1-(2-thienyl)-1-propanone. This reaction step is preferably carried out at a temperature of 20 to 150° C., particularly preferably at 40 to 120° C. and very particularly preferably at 50 to 90° C., and at a pressure preferably of 0 to 10 bar and particularly preferably of 0 to 2 bar. The reaction time is preferably 0.5 to 10 h, particularly preferably 0.5 to 6 h and very particularly preferably 0.5 to 4 h.

The debenzylation step proceeds with an unexpectedly high selectivity, preferably of at least 95%, i.e. the methyl group is barely attacked at all.

The carbonyl group of the thiophene derivative of formula I is reduced in the next reaction step. The reduction is preferably carried out with complex hydrides, e.g. sodium borohydride, lithium borohydride, sodium cyanoborohydride or lithium aluminium hydride, or boranes or mixtures thereof, in a solvent suitable for this purpose, preferably water, methanol, ethanol, isopropyl alcohol, THF, dioxane, diglyme, methylene chloride, toluene, xylene, dichlorobenzene, butyl acetate, ethyl acetate or mixtures thereof, at a temperature conventionally of −20 to 80° C. and preferably at 0 to 25° C.

The reaction pressure is in the range preferably of 0 to 50 bar and particularly preferably of 0 to 5 bar. The molar ratio of thiophene derivative of formula I to complex hydride is conventionally 1:0.25 to 1:4; it is particularly preferable to use equimolar amounts. The reaction time is preferably 0.3 to 10 h.

However, the reduction of the thiophene derivative of formula I to the thiophene derivative containing hydroxyl groups of formula IIa can also be carried out with hydrogen in the presence of a suitable catalyst of homogeneous or heterogeneous type, preferably a metal catalyst such as palladium, platinum, ruthenium, rhodium, nickel or mixtures thereof, in a suitable solvent, preferably methanol, ethanol, isopropyl alcohol, butanol, acetic acid, water, THF, dioxane or mixtures thereof, at a pressure of 1 to 50 bar and preferably of 1 to 20 bar, and at a temperature of 10 to 120° C. and preferably of 20 to 70° C. The catalysts can optionally be attached to suitable supports.

In the last reaction step the carbamate group of the thiophene derivative of formula IIa is cleaved by hydrolysis. The hydrolysis, which is preferably carried out with a conventionally equimolar amount of alkali metal hydroxides, alkaline earth metal hydroxides, alkali metal carbonates, alkali metal hydrogencarbonates, ammonia or mixtures thereof, in a suitable solvent, preferably water, methanol, ethanol, isopropyl alcohol or mixtures thereof, at a temperature of 30 to 120° C. and preferably at 50 to 110° C., and under a pressure of 0 to 50 bar and preferably of 0 to 5 bar, yields N-methyl-3-hydroxy-3-(2-thienyl)propylamine after acidification.

As described previously, only the (S) enantiomer of N-methyl-3-hydroxy-3-(2-thienyl)propylamine is suitable as an entity for the synthesis of duloxetine.

One possible way of obtaining optically active N-methyl-3-hydroxy-3-(2-thienyl)propylamine is direct enantioselective synthesis. Thus, in the reduction step in which the thiophene derivative of formula I is converted to the thiophene derivative of formula IIa, it is possible to use complex hydrides in the presence of molar or even catalytic amounts of chiral non-racemic ligands. Examples of suitable chiral non-racemic ligands are amines, amino alcohols, amino acids, alcohols, binaphthols, carboxylic acids, tartaric acid or derivatives thereof, or sugar derivatives. An alternative is to use chiral non-racemic β-chlorodiisopino-camphorylborane or oxazaborolidines based on proline according to Corey's method. In all cases the reaction involves an asymmetric induction to give an enantiomer of the thiophene derivative IIa rather than the racemate formed when using the conventional reducing agents. The reaction temperature in the enantioselective reduction is preferably −80 to 50° C. and particularly preferably −50 to 30° C.

Preference is given to enantioselective reduction using oxazaborolidines based on proline according to Corey's method, which is also known as the CBS reaction and denotes the enantioselective reduction of ketones with boranes in the presence of substoichiometric amounts of an enantiomer of the oxazaborolidine indicated below (J. Am. Chem. Soc. 1987, 109, 5551, J. Am. Chem. Soc. 1987, 109, 7925, J. Org. Chem. 1988, 53, 2861 and Tetrahedron Lett. 1989, 30, 5547).

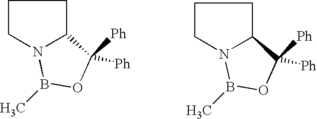

This type of reduction is widely applied to the enantioselective reduction of prochiral ketones to give an alcohol of predicted absolute configuration. The CBS reduction of the N-carbamate-protected ketone of formula I yields the N-carbamate-protected alcohol of formula IIa with ee values preferably of over 85%.

Another possible way of obtaining a desired enantiomer of N-methyl-3-hydroxy-3-(2-thienyl)propylamine is to resolve the racemate of N-methyl-3-hydroxy-3-(2-thienyl)propylamine. This is carried out by reacting racemic N-methyl-3-hydroxy-3-(2-thienyl)propylamine in substoichiometric or equimolar amounts with suitable optically active acids, preferably camphorsulfonic acid, camphoric acid, N-protected amino acids, mandelic acid, malic acid, tartaric acid, O,O′-dibenzoyltartaric acid, glucuronic acid or ascorbic acid, in solvents suitable for the purpose, preferably water, methanol, ethanol, isopropyl alcohol, acetone, methyl isobutyl ketone, ethyl acetate, butyl acetate, methyl tert-butyl ether or mixtures thereof, at a temperature preferably of −30 to 100° C. and particularly preferably of 0 to 30° C. Diastereoisomeric salts of different solubilities are formed. Often only one diastereoisomer crystallizes out and can easily be separated off. N-methyl-3-hydroxy-3-(2-thienyl)propylamine can be liberated from this diastereoisomer with a base, preferably an alkali metal hydroxide, alkaline earth metal hydroxide, alkali metal carbonate, alkali metal hydrogencarbonate, ammonia or mixtures thereof. The liberated, enantiomerically enriched N-methyl-3-hydroxy-3-(2-thienyl)propylamine can be crystallized from a suitable solvent, preferably methanol, ethanol, isopropyl alcohol, water, ethyl acetate, butyl acetate, methyl tert-butyl ether, methyl isobutyl ketone, hexane or mixtures thereof, at a temperature preferably of −30 to 70° C. and particularly preferably of −10 to 30° C., with an enantiomeric excess preferably of at least 95%.

The (S)—N-methyl-3-hydroxy-3-(2-thienyl)propylamine prepared as described above can be used as a starting compound for the synthesis of duloxetine. The preparation of N-methyl-3-hydroxy-3-(2-thienyl)propylamine according to the invention takes place in high yield without the use of expensive reagents and is therefore of great economic value.

The thiophene derivatives of formula II in which R²≠hydrogen are also useful for the synthesis of duloxetine. They are formed by esterification of the alcoholic thiophene derivative IIa with the appropriate carbonyl halides, carboxylic anhydrides or sulfonyl chlorides in a suitable solvent, preferably methylene chloride, 1,2-dichlorobenzene, pyridine, THF, diglyme, methyl tert-butyl ether, toluene, xylene, ethyl acetate, dimethylformamide, dimethylacetamide, dimethyl sulfoxide or mixtures thereof, in the presence of suitable auxiliary bases, preferably pyridine, triethylamine, diisopropylethylamine, 1,4-diazabicyclo[2.2.2]octane (DABCO®), 1,5-diazabicyclo[4.3.0]non-5-ene (DBU), 4-(N,N-dimethylamino)pyridine, sodium carbonate, potassium carbonate, sodium hydrogencarbonate or mixtures thereof. Thiophene derivatives of formula II in which R²≠hydrogen can be reacted, in a solvent suitable for the purpose, either with alkali metal hydroxides to give the corresponding inverted thiophene derivatives of formula IIa, or with naphtholate directly to give the corresponding inverted carbamate-protected N-methyl-3-(1-naphthyloxy)-3-(2-thienyl)propylamine of formula V. This reaction is of particular importance for converting the unwanted enantiomer of N-methyl-3-hydroxy-3-(2-thienyl)propylamine to the desired (S) enantiomer by a Walden inversion and affords an increase in the yield of duloxetine. The preparation and further reaction of the thiophene derivative of formula II are illustrated in Scheme 2.

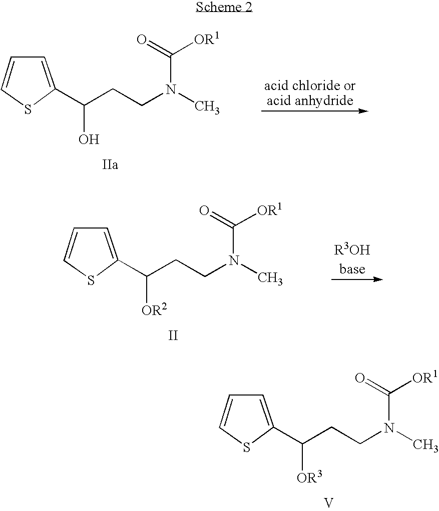

R³ in formula V is either an H atom, in which case the compound is again a thiophene derivative of formula IIa, or the naphthyl radical.

The present invention will now be illustrated in greater detail with the aid of some Examples.

### EXAMPLES 1 to 4

### Preparation of Thiophene Derivatives of Formula I

In the first step, 3-N-benzylmethylamino-1-(2-thienyl)-1-propanone hydrochloride is prepared from 2-acetylthiophene by aminomethylation with N-benzylmethylamine according to EP 457 559 (Example 2).

### Example 1

3-N-methoxycarbonyl-N-methylamino-1-(2-thienyl)-1-propanone

125.4 g (0.42 mol) of 3-N-benzylmethylamino-1-(2-thienyl)-1-propanone hydrochloride and 88.9 g (1.06 mol) of sodium hydrogencarbonate are suspended in 330 ml of toluene, and 59.5 g (0.63 mol) of methyl chloroformate are added. The suspension is refluxed for 3 h. After cooling to room temperature, the precipitate is filtered off with suction and the clear filtrate is concentrated under vacuum to leave 81.5 g (85%) of a clear yellowish oil.

¹H NMR (DMSO-d₆): 2.85 ppm (s, 3H, N—CH₃); 3.25 ppm (t, 2H, CO—CH₂); 3.55 ppm (t, 2H, N—CH₂); 3.58 ppm (s, 3H, OCH₃); 7.22 ppm (t, 1H, aromat.); 7.96 ppm (dd, 2H, aromat.).

### Example 2

3-N-ethoxycarbonyl-N-methylamino-1-(2-thienyl)-1-propanone

465.7 g (1.57 mol) of 3-N-benzylmethylamino-1-(2-thienyl)-1-propanone hydrochloride and 330.1 g (3.93 mol) of sodium hydrogencarbonate are suspended in 1.3 l of toluene, and 256.1 g (2.36 mol) of ethyl chloroformate are added. The suspension is refluxed for 3 h. After cooling to room temperature, the precipitate is filtered off with suction and the clear filtrate is concentrated under vacuum to leave 346 g (91%) of a clear oil. The product distils at 170° C./0.01 Torr.

¹H NMR (DMSO-d₆): 1.15 ppm (t, 3H, ester CH₃); 2.81 ppm (s, 3H, N—CH₃); 3.18 ppm (t, 2H, CO—CH₂); 3.55 ppm (broad, 2H, N—CH₂); 3.98 ppm (broad, 2H, OCH₂); 7.25 ppm (t, 1H, aromat.); 7.98 ppm (dd, 2H, aromat.).

### Example 3

3-N-isobutoxycarbonyl-N-methylamino-1-(2-thienyl)-1-propanone

29.6 g (0.1 mol) of 3-N-benzylmethylamino-1-(2-thienyl)-1-propanone hydrochloride and 21 g (0.25 mol) of sodium hydrogencarbonate are suspended in 100 ml of toluene, and 26.5 g (0.15 mol) of isobutyl chloroformate are added. The suspension is refluxed for 3 h. After cooling to room temperature, the precipitate is filtered off with suction and the clear filtrate is concentrated under vacuum to leave 29.8 g (95%) of a clear yellowish oil.

¹H NMR (DMSO-d₆): 0.86 ppm (s, 6H, 2×CH₃); 1.85 ppm (s, 1H, isopropyl CH); 2.86 ppm (s, 3H, N—CH₃); 3.25 ppm (t, 2H, CO—CH₂); 3.55 ppm (broad, 2H, N—CH₂); 3.72 ppm (broad, 2H, OCH₂); 7.21 ppm (t, 1H, aromat.); 7.95 ppm (dd, 2H, aromat.).

### Example 4

3-N-benzyloxycarbonyl-N-methylamino-1-(2-thienyl)-1-propanone

29.6 g (0.1 mol) of 3-N-benzylmethylamino-1-(2-thienyl)-1-propanone hydrochloride and 21 g (0.25 mol) of sodium hydrogencarbonate are suspended in 100 ml of toluene, and 26.9 g (0.15 mol) of benzyl chloroformate are added. The suspension is refluxed for 3 h. After cooling to room temperature, the precipitate is filtered off with suction. The clear filtrate is washed with 50 ml of 2 N HCl, 50 ml of 10% NaOH solution and 50 ml of water and dried over sodium sulfate. After removal of the solvent under vacuum, the residue is crystallized from isopropyl alcohol at 0° C. The crystals are filtered off with suction to give 11 g of a colourless powder.

¹H NMR (DMSO-d₆): 2.93 ppm (d, 3H, N—CH₃); 3.15 ppm (s, 2H, CO—CH₂); 3.55 ppm (broad, 2H, N—CH₂); 5.08 ppm (d, 2H, OCH₂); 7.21 ppm (s, 1H, aromat.); 7.30 ppm (m; 5H, aromat.); 7.95 ppm (m, 2H, aromat.).

### EXAMPLES 5 to 7

### Preparation of Thiophene Derivatives of Formula IIa

### Example 5

N-ethoxycarbonyl-N-methyl-3-hydroxy-3-(2-thienyl)propylamine

316 g (1.31 mol) of 3-N-ethoxycarbonyl-N-methylamino-1-(2-thienyl)-1-propanone from Example 2 are placed in 150 ml of isopropyl alcohol and 150 ml of water and cooled to 5° C., and 26 g of sodium borohydride are added over 2 h. The isopropyl alcohol is distilled off under vacuum and the aqueous phase is extracted with 3×250 ml of toluene. The combined organic phases are washed twice with water and dried over magnesium sulfate, and the solvent is removed to leave 336.1 g (98%) of a colourless oil.

¹H NMR (DMSO-d₆): 1.15 ppm (t, 3H, ester CH₃); 1.85 ppm (m, 2H, CO—CH₂); 2.85 ppm (s, 3H, N—CH₃); 3.42 ppm (m, 2H, NH—CH₂); 3.98 ppm (q, 2H, OCH₂); 4.80 ppm (m, 1H, CH); 5.65 ppm (d, 1H, OH); 6.90 ppm (m, 2H, aromat.); 7.36 ppm (m, 1H, aromat.).

### Example 6

(S)—N-ethoxycarbonyl-N-methyl-3-hydroxy-3-(2-thienyl)propylamine

25 ml (25 mmol) of a 1 M solution of BH₃.THF in THF are mixed together with 1.25 ml (1.25 mmol) of a 1 M solution of (R)-2-methyl-CBS-oxazaborolidine in 30 ml of THF and cooled to 0° C., and 6.03 g (25 mmol) of 3-N-ethoxycarbonyl-N-methylamino-1-(2-thienyl)-1-propanone from Example 2, dissolved in 10 ml of THF, are added over 2 h. The reaction mixture is kept at this temperature for a further 3 h, the solvent is removed and 20 ml of 20% potassium hydrogensulfate solution are added. The aqueous phase is extracted with 3×20 ml of toluene, the combined organic extracts are washed once with 20 ml of water and dried and the solvent is removed. The residue is filtered off on silica gel. The corresponding fractions are combined, the solvent is removed and the residue is taken up in 6 ml of isopropyl alcohol/6 ml of water and refluxed for 3 h with 4 g of potassium hydroxide. After cooling, the mixture is acidified to pH 4 with potassium hydrogensulfate solution, the isopropyl alcohol is distilled off and the aqueous phase is adjusted to pH 11 with 2 N NaOH solution and extracted with 3×20 ml of toluene. After washing with water, the organic extracts are dried over sodium sulfate and the solvent is removed to give 2.5 g of (S)—N-ethoxycarbonyl-N-methyl-3-hydroxy-3-(2-thienyl)propylamine with an ee of >96%.

¹H NMR (DMSO-d₆): 1.85 ppm (m, 2H, CO—CH₂); 2.35 ppm (s, 3H, N—CH₃); 2.62 ppm (m, 2H, NH—CH₂); 3.5 ppm (broad, 1H, OH); 4.91 ppm (m, 1H, CH); 6.90 ppm (m, 2H, aromat.); 7.36 ppm (m, 1H, aromat.).

### Example 7

(R)—N-ethoxycarbonyl-N-methyl-3-hydroxy-3-(2-thienyl)propylamine

(R)—N-ethoxycarbonyl-N-methyl-3-hydroxy-3-(2-thienyl)-propylamine is prepared analogously to Example 6 except that the (S) enantiomer of 2-methyl-CBS-oxazaborolidine is used. 2.2 g of (R)—N-ethoxycarbonyl-N-methyl-3-hydroxy-3-(2-thienyl)propylamine are obtained with an ee of ˜96%.

¹H NMR (DMSO-d₆): 1.85 ppm (m, 2H, CO—CH₂); 2.35 ppm (s, 3H, N—CH₃); 2.62 ppm (m, 2H, NH—CH₂); 3.5 ppm (broad, 1H, OH); 4.91 ppm (m, 1H, CH); 6.90 ppm (m, 2H, aromat.); 7.36 ppm (m, 1H, aromat.).

### Example 8

Preparation of (R,S)—N-methyl-3-hydroxy-3-(2-thienyl)propylamine

325 g (1.34 mol) of N-ethoxycarbonyl-N-methyl-3-hydroxy-3-(2-thienyl)propylamine from Example 5 are dissolved in 325 ml of isopropyl alcohol, and 325 ml of water and 325 ml of KOH are added. After refluxing for 3 hours, the reaction mixture is cooled to 10° C., 1100 ml of 18% hydrochloric acid are added slowly over 2 h and the mixture is stirred for 45 min. After the addition of 120 g of KOH, the isopropyl alcohol is removed from the reaction mixture. The aqueous phase is extracted with 3×125 ml of toluene and the organic phases are washed twice with water and dried over sodium sulfate. The solvent is removed to leave 214 g (93%) of a yellowish oil, which crystallizes on standing. N-methyl-3-hydroxy-3-(2-thienyl)propylamine can not only be removed from the reaction mixture by a non-extractive method; it can also be crystallized from water after distillation of the isopropyl alcohol.

¹H NMR (DMSO-d₆): 1.85 ppm (m, 2H, CO—CH₂); 2.35 ppm (s, 3H, N—CH₃); 2.62 ppm (m, 2H, NH—CH₂); 3.5 ppm (broad, 1H, OH); 4.91 ppm (m, 1H, CH); 6.90 ppm (m, 2H, aromat.); 7.36 ppm (m, 1H, aromat.).

### EXAMPLES 9 to 11

### Resolution of the Racemate of (R,S)—N-methyl-3-hydroxy-3-(2-thienyl)propylamine

### Example 9

(S)—N-methyl-3-hydroxy-3-(2-thienyl)-propylamine.(S)-(+)-mandelate

214 g (1.25 mol) of (R,S)—N-methyl-3-hydroxy-3-(2-thienyl)-propylamine are dissolved in 375 ml of ethyl acetate, and a solution of 110 g (0.72 mol) of L-(+)-mandelic acid in 230 ml of acetone is added. The L-(+)-mandelate salt precipitates out within 10 h at 5° C. After filtration with suction, 169 g (84%) of (S)—N-methyl-3-hydroxy-3-(2-thienyl)propylamine.(S)-(+)-mandelate salt are obtained with an ee value of 74%. After recrystallization from acetone, (S)—N-methyl-3-hydroxy-3-(2-thienyl)propylamine.(S)-(+)-mandelate salt is obtained in the form of a colourless powder.

¹H NMR (DMSO-d₆): 1.72 ppm (m, 2H, CO—CH₂); 2.25 ppm (s, 3H, N—CH₃); 2.30 ppm (s, 2H, OH, NH); 2.65 ppm (m, 2H, NH—CH₂); 4.30 ppm (s, 1H, mandelate CH); 4.65 ppm (m, 1H, CH); 6.69 ppm (d, 1H, aromat.); 6.72 ppm (m, 1H, aromat.); 6.90 ppm (m, 1H, aromat.); 6.99 ppm (m, 2H, aromat.); 7.15 ppm (m, 1H, aromat.).

### Example 10

(R)—N-methyl-3-hydroxy-3-(2-thienyl)-propylamine.(R)-(−)-mandelate

21.4 g (0.125 mol) of (R,S)—N-methyl-3-hydroxy-3-(2-thienyl)propylamine are dissolved in 375 ml of ethyl acetate, and a solution of 11 g (0.072 mol) of D-(−)-mandelic acid in 230 ml of acetone is added. The R-(−)-mandelate salt precipitates out within 10 h at 5° C. After suction filtration, 17 g (84%) of (R)—N-methyl-3-hydroxy-3-(2-thienyl)propylamine.(R)-(−)-mandelate salt are obtained with an ee value of 75%. After recrystallization from acetone, (R)—N-methyl-3-hydroxy-3-(2-thienyl)propylamine.(R)-(−)-mandelate salt is obtained in the form of a colourless powder.

¹H NMR (DMSO-d₆): 1.70 ppm (m, 2H, CO—CH₂); 2.20 ppm (s, 3H, N—CH₃); 2.33 ppm (s, 2H, OH, NH); 2.65 ppm (m, 2H, NH—CH₂); 4.32 ppm (s, 1H, mandelate CH); 4.65 ppm (m, 1H, CH); 6.69 ppm (d, 1H, aromat.); 6.75 ppm (m, 1H, aromat.); 6.92 ppm (m, 1H, aromat.); 6.99 ppm (m, 2H, aromat.); 7.10 ppm (m, 1H, aromat.).

### Example 11

(S)—N-methyl-3-hydroxy-3-(2-thienyl)propylamine

Aqueous KOH solution is added to 167 g of (S)—N-methyl-3-hydroxy-3-(2-thienyl)propylamine.(S)-(+)-mandelate and the mixture is extracted 3 times with toluene. The combined organic extracts are washed once with water and once with saturated NaCl solution. After removal of the solvent, the residue is crystallized first from a 10% solution of ethanol in water and then from ethyl acetate to give 78 g of a colourless powder with a melting point of 72-73° C. and an optical rotation of −12.4 (c=4.4, EtOH). The ee is 98% according to HPLC and capillary electrophoresis.

¹H NMR (DMSO-d₆): 1.72 ppm (m, 2H, CO—CH₂); 2.25 ppm (s, 3H, N—CH₃); 2.65 ppm (m, 2H, NH—CH₂); 3.5 ppm (broad, 2H, NH, OH); 4.65 ppm (m, 1H, CH); 6.69 ppm (d, 1H, aromat.); 6.72 ppm (m, 1H, aromat.); 6.90 ppm (m, 1H, aromat.); 6.99 ppm (m, 2H, aromat.); 7.15 ppm (m, 1H, aromat.).

### Example 12

Preparation of a Thiophene Derivative of Formula II in Which R³≠H: N-ethoxycarbonyl-N-methyl-3-acetoxy-3-(2-thienyl)propylamine

A mixture of 6.32 g (2.62 mmol) of N-methyl-3-hydroxy-3-(2-thienyl)propylamine and 50 ml of acetic anhydride is heated at 110° C. for 2 h. The acetic anhydride is removed from the reaction mixture under an oil pump vacuum. The residue is taken up in 50 ml of toluene, washed with 2×20 ml of NaOH solution and 2×20 ml of water and dried over sodium sulfate and the solvent is removed to leave 5.9 g of a yellowish oil.

1. Thiophene derivative of formula I: 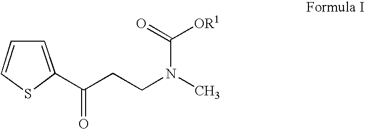
in which R¹ is selected from hydrogen; aliphatic, cycloaliphatic and aromatic hydrocarbon groups which can be substituted by one or more substituents containing heteroatoms; mixed aliphatic-cycloaliphatic hydrocarbon groups which can be substituted by one or more substituents containing heteroatoms; mixed aliphatic-aromatic hydrocarbon groups which can be substituted by one or more substituents containing heteroatoms; and mixed cycloaliphatic-aromatic hydrocarbon groups which can be substituted by one or more substituents containing heteroatoms.

2. Thiophene derivative of formula II: 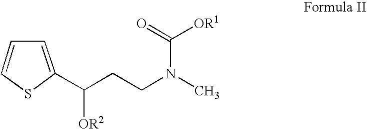
in which R¹ is selected from hydrogen; aliphatic, cycloaliphatic and aromatic hydrocarbon groups which can be substituted by one or more substituents containing heteroatoms; mixed aliphatic-cycloaliphatic hydrocarbon groups which can be substituted by one or more substituents containing heteroatoms; mixed aliphatic-aromatic hydrocarbon groups which can be substituted by one or more substituents containing heteroatoms; and mixed cycloaliphatic-aromatic hydrocarbon groups which can be substituted by one or more substituents containing heteroatoms; and R² is selected from hydrogen; branched or unbranched, saturated or unsaturated acyl groups which can be substituted by one or more substituents containing heteroatoms; aromatic acyl groups which can be substituted by one or more substituents containing heteroatoms; sulfonylalkyl groups; sulfonylalkenyl groups; and sulfonylaryl groups.

3. Thiophene derivative of formula II according to claim 2, characterized in that R² is hydrogen.

4. Thiophene derivative of formula I according to claim 1, characterized in that R¹ is selected from branched or unbranched alkyl groups; branched or unbranched alkenyl groups; branched or unbranched alkynyl groups; halogen-, nitro- or alkoxy-substituted alkyl groups; halogen-, nitro- or alkoxy-substituted alkenyl groups; halogen-, nitro- or alkoxy-substituted alkynyl groups; a benzyl group; a halogen-, nitro- or alkoxy-substituted benzyl group; a phenyl group; a halogen-, nitro- or alkoxy-substituted phenyl group; a fluorenylalkyl group; a menthyl, fenchyl or cholesteryl group; and a hydroxybornyl group.

5. Thiophene derivative of formula I according to claim 4, characterized in that R¹ is selected from the methyl, ethyl, propyl, benzyl, phenyl, isobutyl and menthyl radicals.

6. Process for the preparation of the thiophene derivative of formula I according to claim 1, comprising the aminomethylation of 2-acetylthiophene with N-methyl-benzylamine and formaldehyde in the presence of an acid to form 3-N-benzylmethylamino-1-(2-thienyl)-1-propanone of formula III: 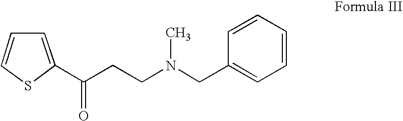
and then the reaction of 3-N-benzylmethylamino-1-(2-thienyl)-1-propanone with a chloroformic acid ester ClCO₂R¹, in which R¹ is as defined in claim 1, in the presence of a base.

7. Process for the preparation of the thiophene derivative of formula II according to, claim 2, by reduction of the thiophene derivative of formula I.

8. Process according to claim 7, characterized in that the reducing agent used is a complex hydride or a borane.

9. Process according to claim 8, characterized in that the complex hydride is selected from sodium borohydride, lithium borohydride, sodium cyanoborohydride, lithium aluminium hydride and mixtures thereof.

10. Process according to claim 8, characterized in that a complex hydride is used as the reducing agent in the presence of a chiral non-racemic ligand, and an optically active thiophene derivative of formula II, is formed by asymmetric induction.

11. Process according to claim 8, characterized in that boranes are used as the reducing agent in the presence of substoichiometric amounts of an enantiomer of the following oxazaborolidine: 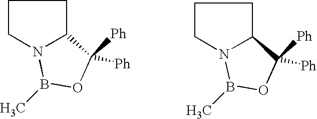
and an optically active thiophene derivative of formula II, is formed by asymmetric induction.

12. Process for the preparation of N-methyl-3-hydroxy-3-(2-thienyl) propylamine of formula IV: 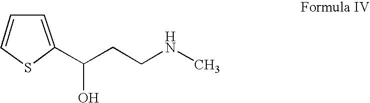
by hydrolysis of the thiophene derivative of formula II according to claim 2.

13. Process according to claim 12, characterized in that an optically active thiophene derivative of formula II is hydrolyzed and optically active N-methyl-3-hydroxy-3-(2-thienyl)-propylamine is formed.

14. Process according to claim 12, characterized in that, after the hydrolysis, the racemate is resolved by reacting N-methyl-3-hydroxy-3-(2-thienyl)propylamine with a suitable optically active acid, the diastereoisomeric salts formed are separated and N-methyl-3-hydroxy-3-(2-thienyl)propylamine is liberated by reacting the desired diastereoisomeric salt with a base to give optically active N-methyl-3-hydroxy-3-(2-thienyl) propylamine.

15. A method for the synthesis of N-methyl-3-(1-naphthyloxy)-3-(2-thienyl) propylamine comprising utilizing the thiophene derivative of claim 1.

16. Thiophene derivative of formula II according to claim 2, characterized in that R¹ is selected from branched or unbranched alkyl groups; branched or unbranched alkenyl groups; branched or unbranched alkynyl groups; halogen-, nitro- or alkoxy-substituted alkyl groups; halogen-, nitro- or alkoxy-substituted alkenyl groups; halogen-, nitro- or alkoxy-substituted alkynyl groups; a benzyl group; a halogen-, nitro- or alkoxy-substituted benzyl group; a phenyl group; a halogen-, nitro- or alkoxy-substituted phenyl group; a fluorenylalkyl group; a menthyl, fenchyl or cholesteryl group; and a hydroxybornyl group.

17. Thiophene derivative of formula II according to claim 16, characterized in that R¹ is selected from the methyl, ethyl, propyl, benzyl, phenyl, isobutyl and menthyl radicals.

18. A method for the synthesis of N-methyl-3-(1-naphthyloxy)-3-(2-thienyl) propylamine comprising utilizing the thiophene derivative of claim 2.
